(19) United States
(12) Patent Application Publication
(10) Pub. No.: US 2021/0196481 A1
(43) Pub. Date: Jul. 1, 2021

(54) COVER DEVICE AND METHOD OF APPLYING COVER DEVICE FOR CONSTRUCTING AND PROTECTING A NIPPLE/AREOLA COMPLEX

(71) Applicants: Ivor Barry Kaplan, Norfolk, VA (US); Michael Wade Dahl, Yorktown, VA (US)

(72) Inventors: Ivor Barry Kaplan, Norfolk, VA (US); Michael Wade Dahl, Yorktown, VA (US)

(21) Appl. No.: 17/181,789

(22) Filed: Feb. 22, 2021

Related U.S. Application Data
(63) Continuation-in-part of application No. 15/890,430, filed on Feb. 7, 2018, now Pat. No. 10,925,719.
(60) Provisional application No. 62/456,869, filed on Feb. 9, 2017.

(51) Int. Cl. A61F 2/52

(52) U.S. Cl. CPC A61F 2/52; A61F 2002/5001; A61F 2002/526

(57) ABSTRACT

A cover device may include a body with an inner surface and an outer surface, each of the inner surface and the outer surface extending from a first end face of the body to a second end face of the body. The inner surface may define a channel extending through the body from the first end face to the second end face. The body may define a plurality of grooves extending from the first end face to the second end face. Portions of each groove may be defined by the inner surface, the first end face, the outer surface, and the second end face. Each of the body, the inner surface, and the outer surface may be configured to elastically deform relative to at least an axis of the channel.

This application is a Continuation-in-part of U.S. patent application Ser. No. 15/890,430 entitled “COVER DEVICE AND METHOD OF APPLYING COVER DEVICE FOR CONSTRUCTING AND PROTECTING A NIPPLE/AREOLA COMPLEX”, filed Feb. 7, 2018, which claims the benefit of priority under 35 U.S.C. § 119(e) to U.S. provisional patent application No. 62/456,869 entitled COVER DEVICE AND METHOD OF APPLYING COVER DEVICE FOR CONSTRUCTING AND PROTECTING A NIPPLE/AREOLA COMPLEX”, filed Feb. 9, 2017, both of which are incorporated by reference in their entireties for all purposes.

### TECHNICAL FIELD

The present disclosure relates to a device and method for constructing and protecting surgically reconstructed tissue structures. More specifically, the present disclosure relates to a device that can be utilized to construct a protruding tissue structure, such as a nipple of a nipple/areola complex, and protect such protruding tissue structure once securing elements, such as sutures, have been removed.

### BACKGROUND

Breast reconstruction may follow breast removal (mastectomy) of a breast that is diseased or has suffered irreparable trauma. There are a number of different methods used for breast reconstruction, one of which may involve the insertion of a permanent breast implant either directly at the time of breast removal, or at a later date when a temporary spacer device (tissue expander) placed under retained breast skin is removed. Other reconstruction methods may involve a transfer of tissue from other sites on a body of a patient to the breast to recreate a breast mound. In a majority of breast removals, a nipple and areola complex (hereafter referred to as “NAC”) is removed with breast tissue, but a large portion or all of the skin of the breast is retained (so called “skin sparing mastectomy”). Accordingly, a majority of breast reconstruction procedures involve nipple and areola reconstruction (NAC reconstruction), regardless of the breast reconstruction method employed.

NAC reconstruction follows an initial creation (reconstruction) of the breast mound and can be achieved in a number of different ways. A realistic NAC reconstruction aids in achieving a realistic looking breast, and may provide tangible psychological benefits to a patient undergoing breast reconstruction. Accordingly, nipple size, projection, position, shape, and color are key considerations in the NAC reconstruction process. Although NAC reconstruction may be done by artful tattooing alone, the most realistic NAC reconstructions often involve: (1) a surgical production of a small skin/fat protuberance (a reconstructed nipple); and (2) a production of a circular flat surface (a reconstructed areola) surrounding the reconstructed nipple that is usually of a different color and/or texture from surrounding breast skin (as is normally present in the intact breast).

The reconstructed nipple may be formed from small flaps of skin (e.g. a skate flap, S flap, C-V flap, star flap, etc.) (e.g. skate flaps or S flaps) elevated from a surface of the breast and rearranged and sewn together in such a way as to produce a projecting neo nipple. The reconstructed areola may be created by subsequent tattooing of the surrounding breast skin or formally created by a surgical application of a skin graft placed around the fabricated central projecting nipple construct (reconstructed nipple).

For NAC reconstruction procedures utilizing skin grafts to reconstruct the areola, it is important to apply pressure to the skin graft in order to facilitate revascularization by, and more importantly attachment with, an underlying bed of tissue. Subsequent to (7 to 10 days) the NAC reconstruction procedure involving the combined nipple and areola reconstructions, pressure must be continuously and evenly applied to the surface of the skin graft to prevent gaps between the bed and the skin graft from developing that may put the skin graft at risk of dying and having to be replaced.

Whether or not a skin graft is employed in the areola reconstruction, the reconstructed nipple is generally formed as mentioned above, from a flap of skin that is part of spared skin of the (mastectomized) breast. Alternatively, the reconstructed nipple may be formed from a part of skin of a flap that has been brought up to a chest of the patient to make the breast mound if implants alone are not utilized for the breast reconstruction. The reconstructed nipple may however, have a tendency to “slump” or become less protruding from the breast if it is not somehow maintained in an upright position relative to the breast. The reconstructed nipple, immediately after a respective reconstruction, is vulnerable and must be protected similar to a skin graft used for an areola reconstruction. For example, tensile/pulling forces from scar tissue formation or putting on articles of clothing, such as garments and breast support devices (e.g. bra), or slipping bandages, may pull apart sutures used to construct and hold the nipple together and cause the nipple to structurally fail. Further, the articles of clothing mentioned above can place pressure on and compress the reconstructed nipple, resulting in a flattening thereof and prevention of the reconstructed nipple from obtaining a desired appearance.

Surgeons often fabricate what is known as a “bolster” from cotton soaked in mineral oil and saline, and secure the bolster over the NAC. Using this technique, a reconstructed nipple will be surrounded by cotton wool pledgets soaked in saline and mineral oil and layered to be built up to, or slightly higher than, a height of the reconstructed nipple. Further, the bolster may be secured to the NAC with the intention to apply pressure to the skin graft and protect the reconstructed nipple (the flap of the nipple) from direct pressure during a healing process.

As may be easily understood by one of ordinary skill in the art, a bolster must be arranged very carefully around a nipple reconstructed from a skin flap at a center of an areola reconstructed from a skin graft so as not to compress and damage the reconstructed nipple. In addition, such a handmade bolster device cannot continue to be utilized to protect the NAC in the weeks following surgery. Therefore, many surgeons resort to subsequent improvised handmade devices, for example, by cutting holes in gauze and stacking the gauze one on the other to build up a protective cylinder around the reconstructed nipple. The perforated gauze pads may be stacked to the same height or slightly above the nipple height and then medical tape or clear sticky adhesive plastic sheeting may be applied over the gauze pads and on to the surrounding breast to prevent shifting.

Other methods attempted to protect a reconstructed nipple have included cutting a top end off a plastic syringe barrel with a blade or hot cautery device to a height slightly more than the height of the reconstructed nipple. The barrel of the syringe segment may be placed over the reconstructed nipple with a small flanged edge at a top of the cut off barrel placed on the breast skin around the reconstructed nipple. The cut off barrel may be secured with medical tape or some type of suture arrangement to prevent this cumbersome arrangement from dislodging and crushing the reconstructed nipple. However, like other processes of creating objects by hand, it can be difficult to expediently cut a syringe time after time and obtain uniform results. As a result, there is no guarantee that a first device formed from a syringe barrel will be suitable for use. Thus, in addition to a lack of repeatable options yielding uniformly configured devices that apply pressure to skin grafts, there is also lack of options for easily and reliably protecting a reconstructed nipple after surgery.

The varied techniques mentioned above involve time-consuming and often messy procedures that must be performed on the fly in an operating room (OR).

A dressing such as or similar to the bolster described above, may remain on a patient for several days after the nipple or NAC reconstruction surgery. During a patient follow-up meeting, the reconstructed nipple or NAC may be redressed in a similar fashion for continued protection as was provided in the OR. For a period of several weeks after the surgery, the patient may be instructed to reapply a protective dressing after showering, not to wear a compressive bra, and not to undertake any activities where the sutures and integrity of the reconstructed nipple could be compromised. Thus, the patient may be required to take numerous precautions involving time consuming and difficult redressing of the reconstructed nipple for an appreciable time after surgery.

These and other issues are solved by a cover device and method of applying a cover device for constructing and protecting a nipple/areola complex, of the present disclosure.

### SUMMARY

According to an aspect of the present disclosure, a cover device may include a body with an inner surface and an outer surface, each of the inner surface and the outer surface extending from a first end face of the body to a second end face of the body. The inner surface may define a channel extending through the body from the first end face to the second end face. The outer surface may define a plurality of grooves extending from the first end face to the second end face. According to an aspect of the present disclosure, each of the body, the inner surface, and the outer surface may be configured to elastically deform relative to at least an axis of the channel.

According to an aspect of the present disclosure, a method of constructing and protecting nipple areola complex of a breast includes suturing a skin graft corresponding to an areola of the nipple/areola complex in a de-epithelialized bed surrounding a nipple of the nipple/areola complex. The method may include leaving a plurality of long sutures during the suturing, each of the plurality of long sutures extending over a length from an outer circumference of the skin graft that is equal to at least twice the diameter of the skin graft. According to an aspect of the present disclosure, the method may include positioning a cover device relative to the nipple/areola complex such that the nipple is positioned within a channel defined by an inner surface of the cover device with a cap of the nipple positioned between a first end face and a second end face of the cover device, and the second end face abuts a surface of the skin graft. According to another aspect of the present disclosure, the method further may include positioning each of the plurality of long sutures in a respective groove defined by an outer surface of the cover device, and tying the plurality of long sutures together at the first end face such that the cover is secured to the nipple/areola complex with the second end face in fixed abutment with the surface of the skin graft.

According to another aspect of the present disclosure, a breast support device includes a first cup and a second cup. At least on of the first cup and the second cup may include a padded region extending from a surrounding edge to an intermediate edge, and a continuous elastic region extending from the intermediate edge. According to an aspect of the present disclosure, the continuous elastic region may include a seam that is concentric with the intermediate edge and a plurality of strips provided on an inner cup surface extending from the seam toward a center of the continuous elastic region.

According to a still further aspect of the present disclosure, a cover device includes a body including an inner surface and an outer surface, each of the inner surface and the outer surface extending from a first end face of the body to a second end face of the body. An outer diameter of the first end face may be less than a outer diameter of the second end face, the inner surface may define a channel extending through the body from the first end face to the second end face, and the outer surface may define a plurality of grooves extending from the first end face to the second end face. According to an aspect of the present disclosure, the body may be formed of ethyl vinyl acetate.

### BRIEF DESCRIPTION OF THE DRAWINGS

The disclosure will be best understood through consideration of, and with reference to, the following figures, viewed in conjunction with the Detailed Description, and in which:

FIG. 1 illustrates a perspective view of a breast and a cover device, according to an aspect of the present disclosure.

FIG. 2A illustrates a top perspective view of a cover device, according to an aspect of the present disclosure.

FIG. 2B illustrates a side elevation view of a cover device, according to an aspect of the present disclosure.

FIG. 2C is a top view of a cover device, according to an aspect of the present disclosure.

FIG. 2D is a bottom view of a cover device, according to an aspect of the present disclosure.

FIG. 2E is an enlarged view of a portion of FIG. 2D designated as Detail 2E and includes a view of one groove of a cover device.

FIG. 3A illustrates a process of positioning a cover device on a NAC, according to an aspect of the present disclosure.

FIG. 3B illustrates a process of securing a cover device to a NAC, according to an aspect of the present disclosure.

FIG. 4 illustrates a perspective view of a breast and a cover device, according to an aspect of the present disclosure.

FIG. 5A illustrates a top perspective view of a cover device, according to an aspect of the present disclosure.

FIG. 5B illustrates a side elevation view of a cover device, according to an aspect of the present disclosure.

FIG. 5C is a top view of a cover device, according to an aspect of the present disclosure.

FIG. 5D is a bottom view of a cover device, according to an aspect of the present disclosure.

FIG. 6 is an enlarged view of a portion of FIG. 5D designated as Detail 6 and includes a view of one groove of a cover device.

FIG. 7A illustrates a process of designating a proposed position of a nipple/areola complex (NAC) for a method of constructing and protecting a NAC, according to an aspect of the present disclosure.

FIG. 7B illustrates a process of designating a position of a flap, according to an aspect of the present disclosure.

FIG. 7C illustrates a process of elevating a flap on a pedicle core, according to an aspect of the present disclosure.

FIG. 7D illustrates a process of wrapping a flap around a pedicle core, according to an aspect of the present disclosure.

FIG. 7E illustrates a process of designating additional skin for removal from a breast, according to an aspect of the present disclosure.

FIG. 7F illustrates a process of positioning a skin graft on to an initial de-epithelialized bed, according to an aspect of the present disclosure.

FIG. 7G illustrates a process of suturing a skin graft in an expanded de-epithelialized bed, according to an aspect of the present disclosure.

FIG. 7H illustrates a process of positioning a cover device on a NAC, according to an aspect of the present disclosure.

FIG. 7I illustrates a process of securing a cover device to a NAC, according to an aspect of the present disclosure.

FIG. 8 illustrates a side elevation of a breast with a cover device secured to the breast, according to an aspect of the present disclosure.

FIG. 9 illustrates a cross-sectional view of a breast taken along section line 9-9 of FIG. 8.

FIG. 10A illustrates an overhead view of a proposed position for a NAC, according to an aspect of the present disclosure.

FIG. 10B illustrates front perspective view of a reconstructed NAC, according to an aspect of the present disclosure.

FIG. 11 illustrates a state of a pair of breasts prior to being positioned in a breast support device with a cover device, according to an aspect of the present disclosure.

FIG. 12 illustrates a state of a pair of breasts prior to being positioned in a breast support device with a cover device, according to an aspect of the present disclosure.

FIG. 13 illustrates an elevation view of an inner cup surface of a breast support device, according to an aspect of the present disclosure.

The figures presented are intended solely for the purpose of illustration and they are, therefore, neither desired nor intended to limit the subject matter of the disclosure to any or all of the exact details of construction shown, except insofar as they may be deemed essential to the claims.

### DETAILED DESCRIPTION

Aspects of the disclosure will now be described in detail with reference to the figures, wherein like reference numbers refer to like elements throughout, unless specified otherwise. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Aspects of the present disclosure described herein are directed toward a cover device for reconstructing and protecting a nipple alone or a nipple/areola complex (NAC). The cover device may include a dome shaped body including an inner surface that defines a channel, and an outer surface that defines a plurality of grooves configured to receive sutures. A head of the body may define a first end face that is flat and defines a plurality of notches; each notch may extend from a first groove end of a respective groove. Further, a base of the body may define a second end face that is flat and defines second groove ends for each groove. In addition, the body may be elastically deformable.

The cover device may be positioned on a reconstructed NAC during a nipple reconstruction procedure. The cover device may be secured to the NAC by sutures that are received in respective grooves and tied together at the first end face over the channel, which surrounds a nipple of the NAC. In this configuration, the cover device is deformed by the compressive force of the tied sutures, but maintains a shape of the second end face to transmit the force and evenly apply pressure to the skin graft. The continuous and evenly applied pressure thereby reliably facilitates vascularization and attachment of the skin graft with a de-epithelialized bed formed on a breast. Concurrently, the channel holds the nipple of the NAC in an upright position protruding from the breast and protects the nipple. Once the sutures are removed, the same or new cover device may be positioned on the NAC between a breast support device and the breast and used to protect the NAC (in particular the nipple of the NAC) from being compressed or rubbed by garments worn by a patient.

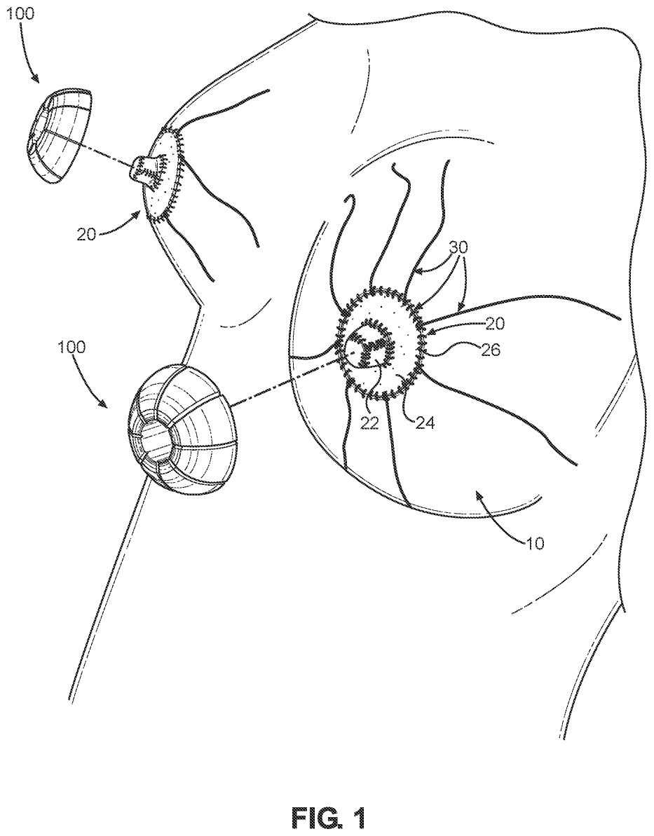

FIG. 1 illustrates a perspective view of a breast 10 and a cover device 100 following a nipple reconstruction procedure, according to an aspect of the present disclosure. The breast 10 includes a nipple/areola complex 20 (hereafter referred to as “NAC 20” or “reconstructed NAC 20”) that has been reconstructed. The NAC 20 may include a nipple 22, and a skin graft 24. The skin graft 24 corresponds to an areola of the reconstructed NAC 20 and may be attached to a circumferential skin edge 26 with sutures 30.

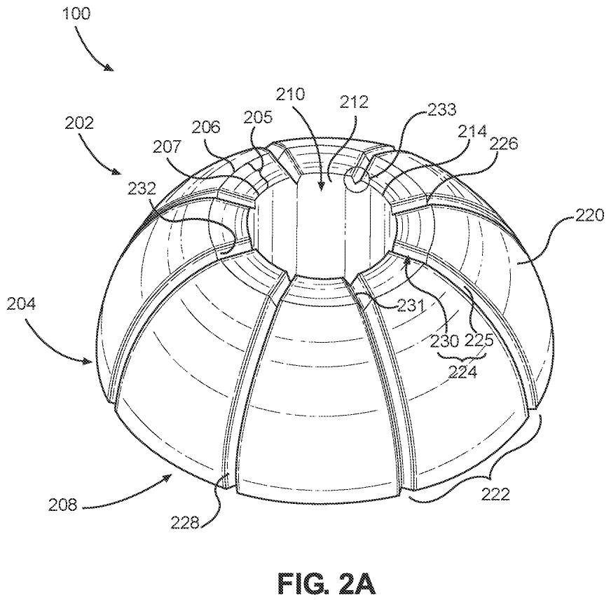

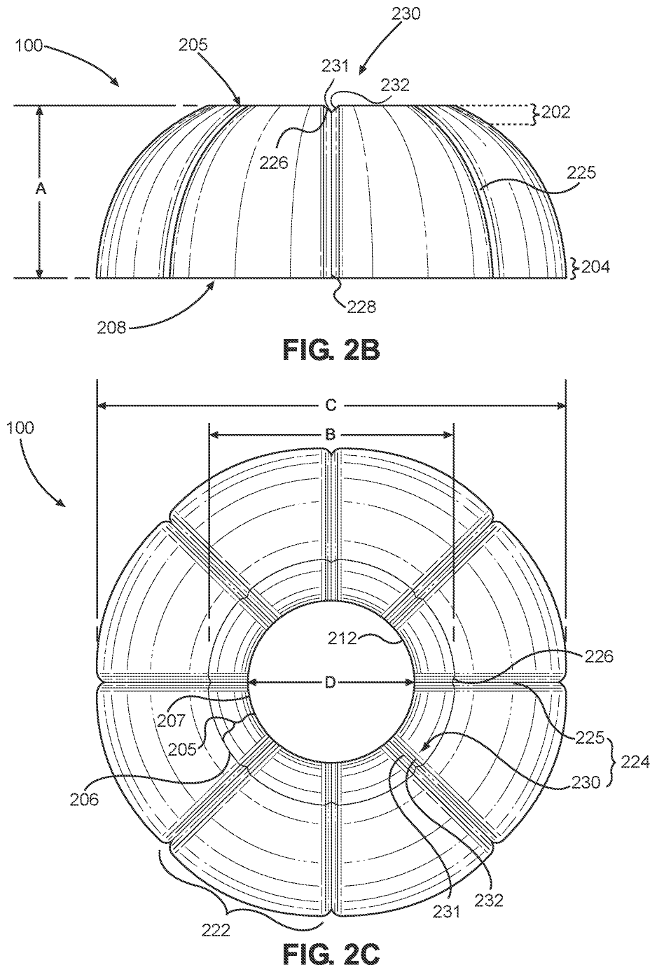

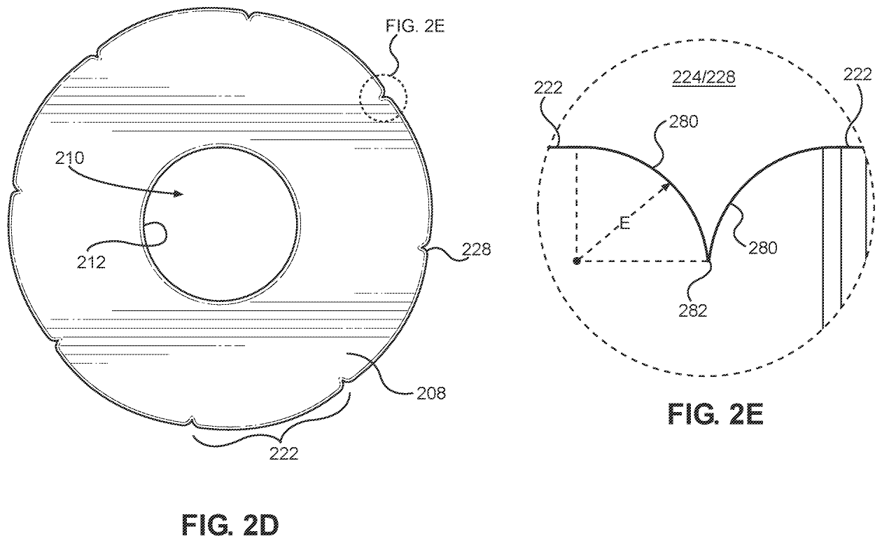

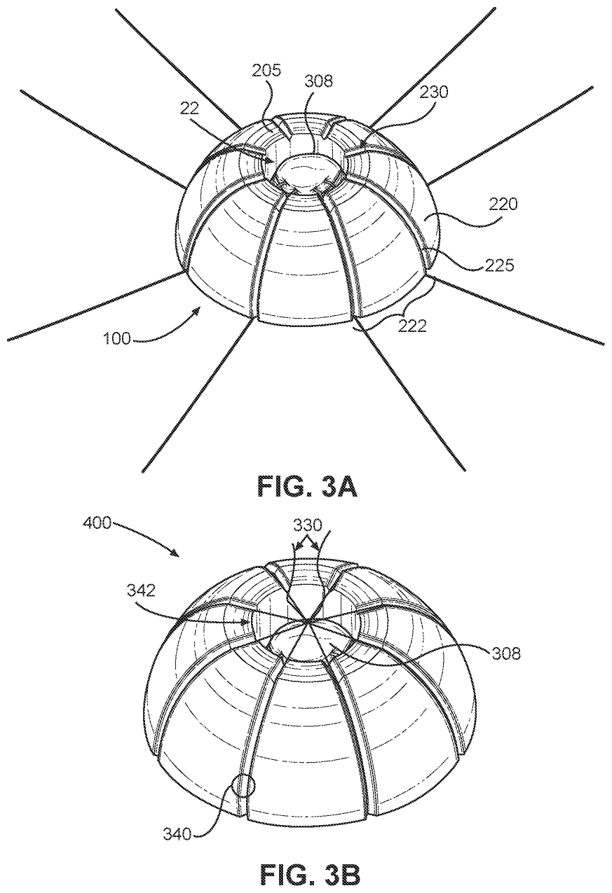

According to one aspect of the present disclosure, the cover device 100 may have a mushroom top or dome-like shape as illustrated in FIG. 1, and as explained in more detail below with reference to FIGS. 2A-2E. Further, the cover device 100 may be secured onto the NAC 20 by some of the sutures 30 (left long for that purpose), with the nipple 22 inserted into the cover device 100 as explained in more detail with reference to FIGS. 3A and 3B.

FIG. 2A illustrates a top perspective view of the cover device 100. The cover device 100 includes a body 200 that is mushroom or dome-like in shape and includes a head 202 and a base 204. The head 202 defines a first end face 205, and the base 204 defines a second end face 208 (see FIG. 2D) of the body 200; each of the first end face 205 and the second end face 208 being flat. A channel 210 is defined by an inner surface 212 of the body 200 and extends between respective apertures 214 defined in the first end face 205 (see FIGS. 2A and 2C) and the second end face 208. Thus, the channel 210 may be in the form of a cylindrical conduit or tube as illustrated in FIG. 2A. A plurality of grooves 224 are defined an outer surface 220, first end face 205, and second end face 208 of the body 200 of the cover device 100. Each groove 224 includes a body groove 225 and a notch 230 as described in more detail below.

The outer surface 220 of the body 200 is composed of a plurality of wall segments 222 as provided by the body grooves 225 defined by/formed in the outer surface 220. Each body groove 225 extends to the second end face 208 from a body groove end 226 at a perimeter edge 206 of the first end face 205. More specifically, each body groove 225 extends from a respective first body groove end 226 formed at the perimeter edge 206, to a second body groove end 228 formed in the second end face 208. A notch 230 of each groove 224 extends inwardly toward the inner surface 212 from each first body groove end 226 at the perimeter edge 206. Each first body groove end 226 thereby serves as a transition between a respective body groove 225 and notch 230 of each groove 224. Each notch 230 terminates in the inner surface 212 at an inner edge 207 of the first end face 205 where a notch end 233 of the notch 230 defines a void within the inner surface 212. A shape of each void is defined by notch walls 232 extending from the first end face 205 to a joint edge 231 where the notch walls 232 converge.

FIG. 2B illustrates a side elevation view of the cover device 100. As shown, the body 200 has a height A that may be in a range of 1.3 to 1.7 cm. According to an aspect of the present disclosure, the height A may be 1.5 cm. FIG. 2B further illustrates a curvature of the wall segments 222 and the body grooves 225 as defined by the shape of the outer surface 220. It will be understood that the body 200, and therefore the outer surface 220, may be in the form of other shapes such as a polygon, a pyramid, a square, etc. More generally, the body 200 may be formed into a shape that facilitates the placement of the sutures 30 within the body grooves 225 and notches 230 of the grooves 224, and tying of the sutures 30 at the first end face 205 to secure the cover device 100 to the NAC 20.

FIG. 2C is a top view of the cover device 100. As shown, the cover device 100 has a first outer diameter B corresponding to a diameter of the perimeter edge 206 of the first end face 205, and a second outer diameter C as defined by a diameter of a perimeter of the base 204. The first outer diameter B may be in an exemplary range of 1.7 to 2.2 cm, and the second outer diameter C may be in a range of 4.3 to 4.7 cm. In one example, the first outer diameter B may be 1.9 cm and the second outer diameter C may be 4.5 cm. In addition, the channel 210 may have a channel diameter D, corresponding to the inner edge 207 of the first end face 205, in an exemplary range of 1.4 to 1.6 cm, and may preferably be 1.5 cm. The ranges for the dimensions discussed herein are exemplary. One of ordinary skill in the art will recognize that dimensions of the cover device 100 may be adjusted to match a size of the NAC 20 desired by a surgeon and/or patient and vice versa.

According to another aspect of the present disclosure, the cover device 100 may be provided in a range of standardized sizes since a surgeon may be able to construct the NAC 20 to be a desired size corresponding to a size of the cover device 100. More specifically, the cover device 100 may be provided in a range of sizes that correspond to, or are slightly larger or smaller than, typical, or desired, or naturally occurring outer diameters of skin grafts used for areolas and reconstructed nipples. Furthermore, the range of standardized sizes may correspond to typical sizes of skin grafts and reconstructed nipples at the time of surgery or post-operation, or even based on typical eventual sizes once healed.

FIG. 2C further illustrates the notches 230 of the grooves 224. Each notch 230 extends: (A) from a respective first body groove end 226 defined by the body 200 at the perimeter edge 206 of the first end face 205, (B) to a respective notch end 233 defined in the inner surface 212 at the inner edge 207 of the first end face 205. According to an aspect of the present disclosure, the curved slope of each body groove 225 transitions at a respective first body groove end 226, to the recessed passage of a respective notch 230 having a relatively linear profile. In one example, each the joint edge 231 is therefore relatively flat and thus runs parallel to a plane of the second end face 208 and perpendicular to a longitudinal axis of the cover device 100. In another example, the joint edge 231 may have a slight angle such that a low point of the joint edge 231 at the first body groove end 226 is lower than the joint edge 231 at the notch end 233—within a frame of reference corresponding to an elevation view of FIG. 2B. With either notch configuration, and variations thereof, the first body groove end 226 and the notch 230 may guide an element, such as a suture 30, (A) from a respective body groove 225 onto a notch surface defined by a joint edge 231, which is formed with the first end face 205 between notch walls 232, and (B) across a plane corresponding to the inner surface 212.

Running between walls 232 that define the notch 230, sutures 30 may have little opportunity to move about a surface of the first end face 205. By keeping the sutures 30 in these bounded positions provided by the body groove 225 and the notch 230 of each groove 224, a surgeon may be able to readily tie opposing sutures 30 together without concern of sutures 30 becoming misaligned. In addition, in contrast to when bolsters are employed, the surgeon can ensure downward forces will be applied evenly by the cover device 100 without having to make several attempts to tie the sutures 30 in such a way that they are evenly distributed about a perimeter that shares an axis with a center of a reconstructed nipple/NAC.

One of ordinary skill in the art will understand that an amount of pressure applied to the skin graft 24 by the second end face 208 is proportional to a magnitude of a downward force normal to a surface of the skin graft 24. According to one aspect of the non-limiting exemplary configuration of the cover device 100 illustrated in FIGS. 1-2E, once the sutures are tied, forces applied by sutures 30 may be directed towards the second end face 208.

FIG. 2D is a bottom view of the cover device 100, according to an aspect of the present disclosure. As illustrated in FIGS. 2B and 2D, the second end face 208 defines a flat annular surface that surrounds the aperture 214 of the channel 210. The flat annular surface may be configured with the second outer diameter C sized to correspond to a desired circumferential skin edge 26 (FIG. 1) or vice versa. Unlike a bolster described above, the flat annular surface of the second end face 208 may apply pressure evenly over the surface area of the surface of the skin graft 24. As a result, the skin graft 24 may be more uniformly pressed against a de-epithelialized bed and more effectively re-vascularized in comparison to a procedure utilizing a device or instrument that is not substantially flat or able to uniformly transmit a downward force applied by a device securing mechanism (e.g., tape, sutures, etc.), such as a bolster.

As further illustrated in FIG. 2D, the second groove ends 228 open on to the flat annular surface of the bottom end face 208.

FIG. 2E is an enlarged view of a portion of FIG. 2D designated as Detail 2E, and includes a view of one second body groove end 228 of the cover device 100. According to an aspect of the present disclosure, the second body groove end 228 and a majority of the body groove 225 including a portion of the first body groove end 226, are defined by groove walls 280 formed/defined by the outer surface 220 of the body 200 as illustrated in FIG. 2E. It will be noted that the groove walls 280 of the body grooves 225 transition from the profile illustrated in FIG. 3 into the notches 230 at first body groove ends 226, which coincide with the perimeter edge 206 at the first end face 205. According to an aspect of the present disclosure, the groove walls 280 are curved surfaces such that a cross-section of the body groove 225 has a curved V-shape. According to another aspect of the present disclosure each groove wall 280 may have a radius of curvature E in an exemplary range of 0.8 to 1.2 mm, and may preferably be 1.0 mm. In one example, the walls 232 of the notches 230 may have the same shape and profile as the groove walls 280. In another example, the walls 232 of the notches 230 and the groove walls 280 of the body grooves 225 may have different shapes.

As illustrated in FIG. 2E, the groove walls 280 extend from a joint edge 282 to an outermost portion of the outer surface 220, which defines the wall segments 222. The groove walls 280 are symmetric about the joint edge 282 that is located at a center of a trench defined by the body groove 225. The cross-section of the body groove(s) 225 is continued over a length of the groove(s) 224 from the second body groove end 228 to the first body groove end 226. As a result, the wall segments 222 may be lobe-shaped as illustrated in FIGS. 2A, 2C, and 2D.

It will be understood that other shapes (e.g., a flat angled wall, a wall extending 90° from a horizontal surface, an inwardly curved or parabolic surface, etc.) may be employed for the walls 232 of the notches 230 and the groove walls 280 of the body grooves 225 such that the grooves 224 have different cross-sectional shapes (e.g. a straight V-shape, a square, a semi-circle, etc.). More generally, the walls 232 and/or the groove walls 280 may have any shape, including a shape that defines the notches 230 and/or the body grooves 225 to have asymmetrical cross-sections, that enable: (A) the body grooves 225 to prevent the sutures 30 from moving radially relative to respective body grooves 225; and (B) the notches 230 to prevent the sutures 30 from moving annularly along the first end face 205 relative to respective notches 230.

According to an aspect of the present disclosure, the body 200 of the cover device 100 may be formed of a material that is elastically deformable. More specifically, the body 200 may be formed from an elastically deformable and/or sponge-like material that is compressible. For example, the cover device 100 may be formed from certain types of thermoplastic materials such as thermoplastic elastomers, ethyl vinyl acetate, or other foam rubber materials. In one example, the cover device may be formed of a thermoplastic elastomer in the form of a thermoplastic elastomer compound, such as STARMED 9025-0000, STARMED 9030-0000, and the like.

More generally, the cover devices described herein may be formed from a thermoplastic material such that an exemplary cover device can fully withstand standard steam sterilization, according to sterilization protocols that may include processes conducted: at temperatures between 121° C. (250° F.) and 134° C. (273° F.); under 15 psi (0.5 bar) pressure; for between 10 and 60 min. In other examples, cover devices described herein may be formed from a thermoplastic material and be able to fully withstand steam sterilization at 121° C. (250° F.) for 30 minutes in a gravity displacement sterilizer, or steam sterilization at 132° C. (270° F.) for 4 minutes in a prevacuum sterilizer.

In various example, the cover device 100 according to the present disclosure may be formed by injection molding, multi-injection molding, blow molding, or from a sheet of elastic material, such as a sheet formed from a thermoplastic elastomer as previously described, or ethyl vinyl acetate. In still other examples, cover devices described herein, including some versions of the exemplary cover device 100, may be cut from a sheet alone, or cut from a sheet of elastic material and further molded into shape.

In addition, versions of exemplary cover devices of the present disclosure that may be formed the thermoplastic materials described herein, may be transparent or semitransparent in appearance. As a result of the cover device being transparent, at least to some degree, a surgeon may be able to assess how a NAC is healing without fully removing the cover device.

FIG. 3A illustrates a process of positioning the cover device 100 on a NAC 20, according to an aspect of the present disclosure. As illustrated in FIG. 3A, a cover device, for example the cover device 100 illustrated in FIGS. 1-2E, is positioned on a skin graft 24 of the NAC 20, and a nipple 22 of the NAC 20 is inserted into the channel 210 of the cover device 100. Prior to positioning the cover device 100 on the NAC 20, the cover device 100, which may have been gamma sterilized, or sterilized according to any of the sterilization protocols mentioned herein or equivalents thereof, is removed from a sterilized packaging. A tip of the nipple 22 is defined by a cap 308, which is sutured to wings of a flap. A location of the cap 308 along an axis of the channel 210 is between the first end face 205 and the second end face 208. More specifically, a height of the nipple 22 may be less than the height A of the cover device 100. Accordingly, the cover device 100 will protrude from the breast 100 past the tip of the nipple 22 and is thus configured to enclose and protect the nipple 22 from external forces (e.g., pressure/rubbing from garments). Furthermore, the shape and height of the channel 210 aids the nipple 22 of the NAC 20 in retaining its shape while healing after surgery.

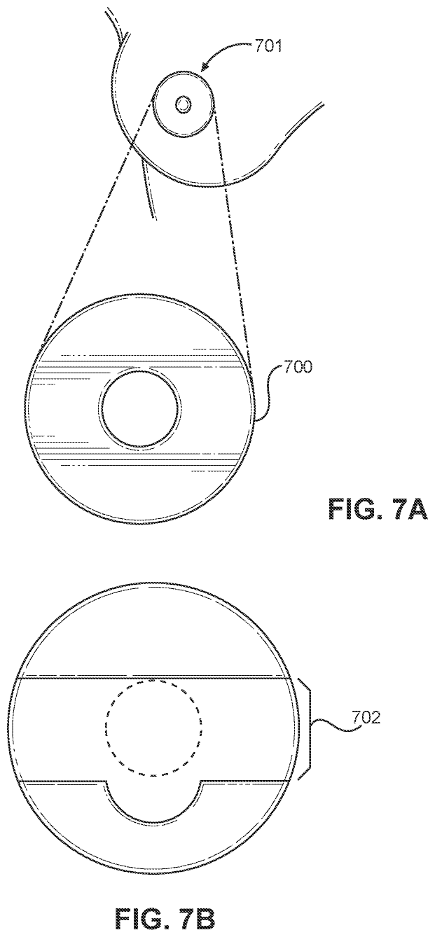

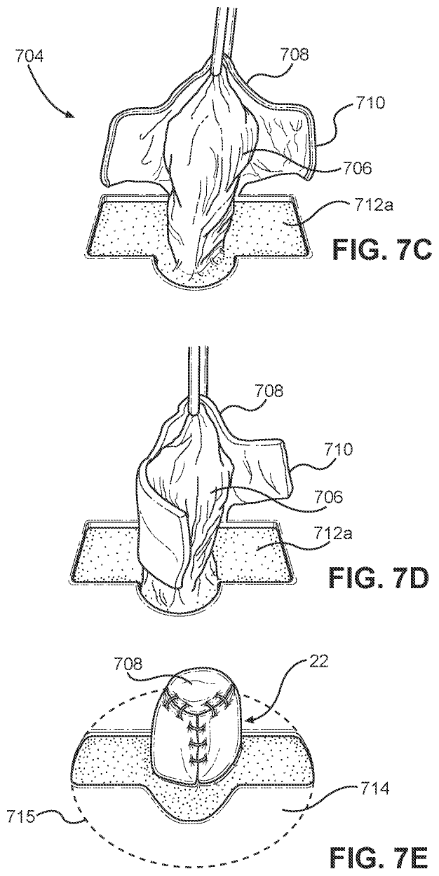

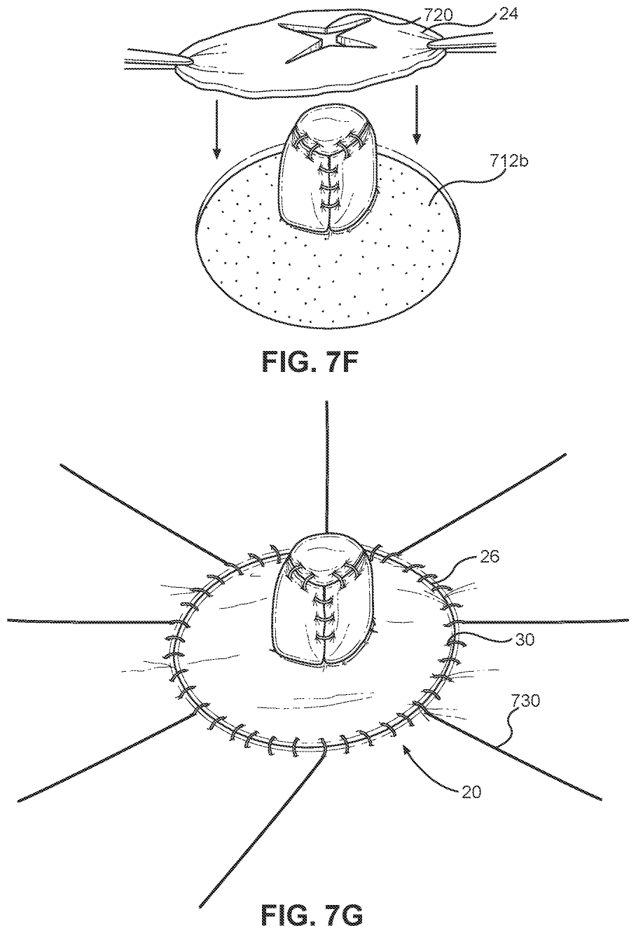

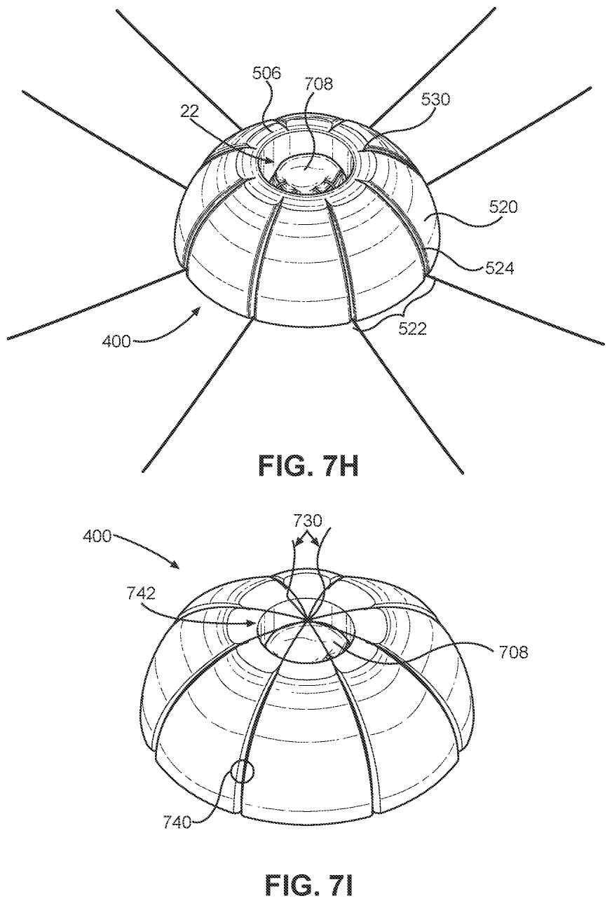

As further illustrated in FIG. 3A, each groove 224 (body groove 225 and notch combination 230) is aligned with a respective long suture 330 extending from the outer diameter of the skin graft 24/circumferential skin edge 26 of a breast 10. A number of long sutures 330 may be equal to a number of the grooves 224. According to an aspect of the present disclosure, there is one long suture 330 for each one of eight grooves 224. However, the cover device 100 may be secured to the NAC as discussed in more detail with reference to FIG. 3B, by more or fewer grooves 224 and corresponding long sutures 330. For example, the cover device 100 may be provided with three, four, or six grooves 224 and a corresponding number of long sutures 330 may be provided during a suturing process, an example of which is illustrated in FIG. 7G.

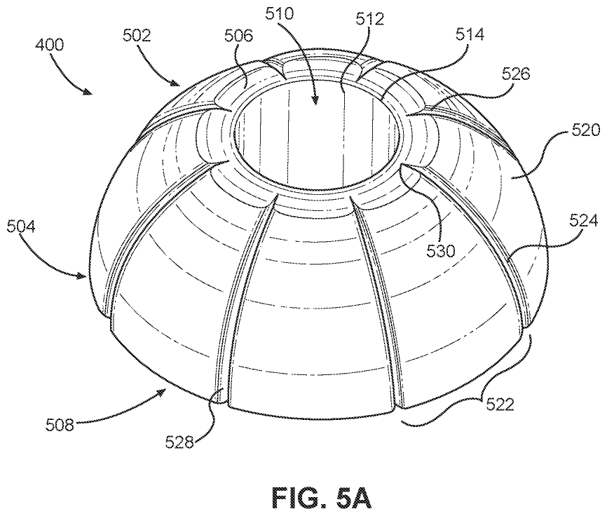

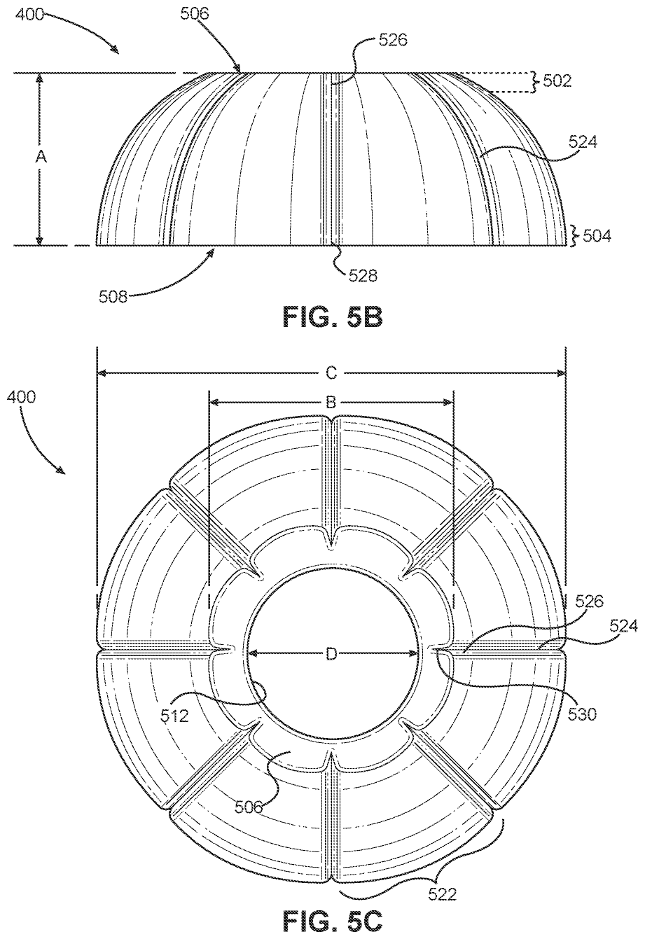

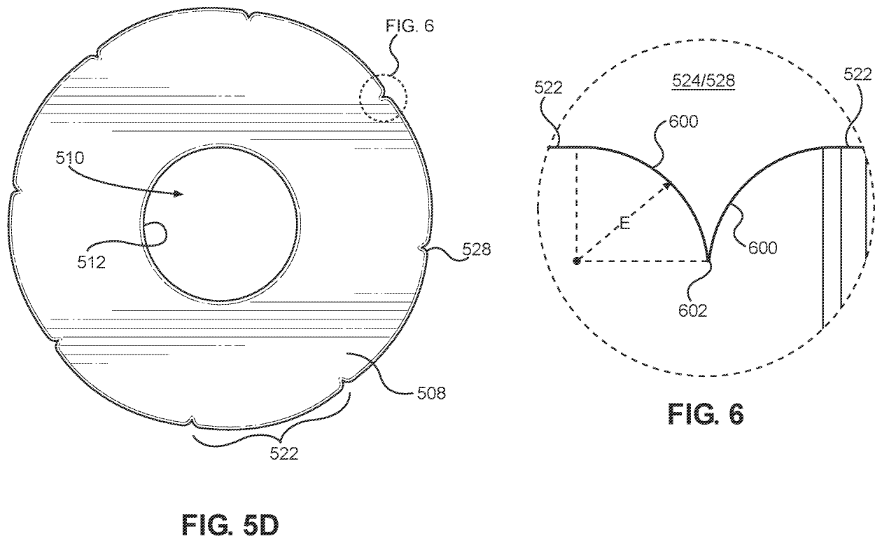

According to another aspect of the present disclosure, the number of long sutures 330 may not have to be equal to the number of grooves 224. The number of grooves 224 and sutures 330 must be such that a provided number of potential grooved suture combinations 340, which are identified in FIG. 3B, is suitable to secure the cover device 100 to the NAC 20 with the second end face 208 pressed against a surface of the skin graft 24 as discussed below for another exemplary cover device of the present disclosure with reference to FIGS. 5 and 6.

FIG. 3B illustrates a plurality of the grooved suture combinations 340 in which a respective long suture 330 has been positioned within a corresponding second body groove end 228, body groove 225, first body groove end 226, and notch 230. Accordingly, the long sutures 330 are positioned in corresponding grooves 224 between the wall segments 222. Further, the long sutures 330 are tied together inwardly relative to the notches 230 in a tie 342, and thereby apply pressure to the first end face 205. More specifically, the long sutures 330 may apply downward forces on the first end face 205 by way of applying forces on the joint edges 231 of the notches 230. The applied forces are cumulatively transmitted through the body 200 to the second end face 208, which applies pressure uniformly to the skin graft 24. Thus, the cover device 100 applies pressure to the skin graft 24 of the NAC 20. At the same time, the head 202, first end face 205, and inner surface 212, together with the tie 342 and portions of the long sutures 330 extending radially between the tie 342 and the first end face 205, protect the nipple 22 of the NAC 20.

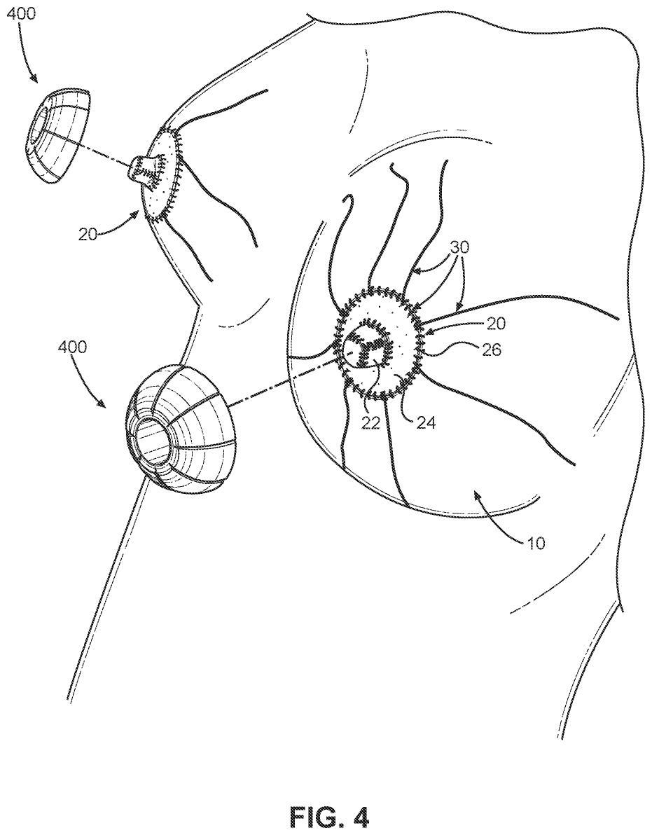

FIG. 4 illustrates a perspective view of a breast 10 and a cover device 400 following a nipple reconstruction procedure, according to an aspect of the present disclosure. The breast 10 includes a nipple/areola complex 20 (hereafter referred to as “NAC 20” or “reconstructed NAC 20”) that has have been reconstructed. The NAC 20 may include a nipple 22, and a skin graft 24. The skin graft 24 corresponds to an areola of the reconstructed NAC 20 and may be attached to a circumferential skin edge 26 with sutures 30.

According to one aspect of the present disclosure, the cover device 400 may have a mushroom top or dome-like shape as illustrated in FIG. 1, and as explained in more detail below with reference to FIGS. 5A-5D. Further, the cover device 400 may be secured onto the NAC 20 by some of the sutures 30 (left long for that purpose), with the nipple 22 inserted into the cover device 400 as explained in more detail with reference to FIGS. 7A-7I.

FIG. 5A illustrates a top perspective view of the cover device 400, according to an aspect of the present disclosure. The cover device 400 includes a body 500 that is mushroom or dome-like in shape and includes a head 502 and a base 504. The head 502 defines a first end face 506, and the base 504 defines a second end face 508 of the body 500; each of the first end face 506 and the second end face 508 being flat. A channel 510 is defined by an inner surface 512 of the body 500 and extends between respective apertures 514 defined in the first end face 506 (FIGS. 5A, 5C) and the second end face 508 (FIG. 5D). Thus, the channel 510 may be in the form of a cylindrical conduit or tube as illustrated in FIG. 5A.

An outer surface 520 of the body 500 is composed of a plurality of wall segments 522 as provided by grooves 524 defined by/formed in the outer surface 520. Each groove 524 extends from the first end face 506 to the second end face 508. More specifically, each groove 524 extends from a first groove end 526 formed in the first end face 526, to a second groove end 528 formed in the second end face 508. Each first groove end 526 extends from a perimeter of the first end face 506 and terminates in the form of a notch 530 in the first end face 506.

FIG. 5B illustrates a side elevation view of the cover device 400, according to an aspect of the present disclosure. As illustrated in FIG. 5B, the body 500 has a height A that may be in a range of 1.3 to 1.7 cm. According to an aspect of the present disclosure, the height A may be 1.5 cm.

FIG. 5B further illustrates a curvature of the wall segments 522 and the grooves 524 as defined by the shape of the outer surface 520. It will be understood that the body 500, and therefore the outer surface 520, may be in the form of other shapes such as a polygon, a pyramid, a square, etc. More generally, the body 500 may be formed into a shape that facilitates the placement of the sutures 30 within the grooves 524, and tying of the sutures 30 at the first end face 506 to secure the cover device 400 to the NAC 20.

FIG. 5C is a top view of the cover device 400, according to an aspect of the present disclosure. As illustrated in FIG. 5C, the cover device 400 has a first outer diameter B corresponding to a diameter of the perimeter of the first end face 506, and a second outer diameter C as defined by a diameter of a perimeter of the base 504. The first outer diameter B may be in a range of 1.7 to 2.2 cm, and the second outer diameter C may be in a range of 4.3 to 4.7 cm. According to an aspect of the present disclosure, the first outer diameter B may be 1.9 cm and the second outer diameter C may be 4.5 cm. In addition, the channel 510 may have a channel diameter D in a range of 1.4 to 1.6 cm, and may preferably be 1.5 cm. The ranges for the dimensions discussed herein are exemplary. One of ordinary skill in the art will recognize that dimensions of the cover device 400 may be adjusted to match a size of the NAC 20 desired by a surgeon and/or patient and vice versa. According to another aspect of the present disclosure, the cover device 400 (or cover device 100) may be provided in a range of standardized sizes since a surgeon may be able to construct the NAC 20 to be a desired size corresponding to a size of the cover device 400 (or cover device 100).

FIG. 5C further illustrates notches 530, each of which extends from a respective first groove end 526 defined by the body 500. According to an aspect of the present disclosure, the curved slope of each groove 524 and first groove end 526, combined with a respective notch 530 defines a continuous transition (e.g. a ramp) from the first groove end 526 to a flat surface of the first end face 506. Accordingly, the first groove end 526 and the notch 530 may guide an element, such as a suture 30, from a respective groove 524 onto the flat surface of first end face 506 without causing the suture 30 to bend or “kink” at a sharp angle (e.g. 90°) as the suture 30 transitions from the groove 524 to the surface of the first end face 506. As a result, when the suture 30 is tied to the other sutures 30, an angle of a direction along which a downward force (i.e. vector) is applied to the first end face 506 by each suture 30 is closer to a direction that is normal to a surface of the skin graft 24. In contrast, a configuration in which a suture 30 bends around a corner at the outer perimeter of the first end face 506 at a sharp angle as described above, may apply a force along a direction that is closer to a direction parallel to the surface of the skin graft 524.

One of ordinary skill in the art will understand that an amount of pressure applied to the skin graft 24 by the second end face 508 is proportional to a magnitude of a downward force normal to a surface of the skin graft 24. According to one aspect of the non-limiting exemplary configuration of the cover device 400 illustrated in FIGS. 4-5D, forces applied by sutures 30 may be directed more towards the second end face 508 (and therefore closer to a direction normal to the surface of the skin graft 24) than the inner surface 512 as a result of the continuous transition provided by the combination of the curvature of the groove 524, the first groove end 526, and the notch 530. This may cause the second end face 508 to be more directly pressed against the surface of the skin graft 24 (i.e., apply more pressure to the skin graft 24) than if the suture 30 was caused to bend around a corner between the first end face 506 and the outer surface 520 at a sharp angle as previously discussed.

However, it will be understood that an optimal amount of pressure that may be applied to a given skin graft of a reconstructed NAC may vary depending on the type of skin graft (e.g. split-thickness, full-thickness) being used. According to one aspect of the present disclosure, a transition between at least the first groove ends 524 (as well as the outer surface 520) and the first end face 506 may be configured according to the optimal pressure to be applied to the surface of the skin graft 24, as dictated by the type of skin graft employed to construct the NAC 20. Accordingly, the present disclosure is not limited to the particular transitions (e.g. ramp) provided by the first groove ends 526 and notches 530 illustrated in FIGS. 4-5D.

In another aspect of the present disclosure, first groove ends and notches may be provided in a configuration that may sacrifice some portion of downward force being applied to a first end face, for ease of use and application during a procedure. Notches and first groove ends could be defined within a cover device based on design constraints that emphasize, in the context of performing a procedure, an ease of: locating a suture within a notch; maintaining a suture within a notch; and using a notch to maintain a suture's alignment and position within a corresponding first groove. An example of such a configuration and its advantages are described above in more detail with reference to FIGS. 1-3B. It will be noted that although some degree of applied pressure may be sacrificed, all the advantages provided with respect to vascularization and taking, for example by the cover device 400, are also provided by the cover device 100 of FIGS. 1-3B, and any other cover device described herein this Detailed Description.

FIG. 5D is a bottom view of the cover device 400, according to an aspect of the present disclosure. As illustrated in FIGS. 5B and 5D, the second end face 508 defines a flat annular surface that surrounds the aperture 514 of the channel 510. The flat annular surface may be configured with the second outer diameter C sized to correspond to a desired circumferential skin edge 26 (FIG. 1) or vice versa. Unlike a bolster described above, the flat annular surface of the second end face 508 may apply pressure evenly over the surface area of the surface of the skin graft 24. As a result, the skin graft 24 may be more uniformly pressed against a de-epithelialized bed and more effectively re-vascularized in comparison to a procedure utilizing a device or instrument that is not substantially flat or able to uniformly transmit a downward force applied by a device securing mechanism (e.g. tape, sutures, etc.), such as a bolster.

As further illustrated in FIG. 5D, the second groove ends 528 open on to the flat annular surface of the bottom end face 508. FIG. 6 is an enlarged view of a portion of FIG. 5D designated as Detail 3 and includes a view of one second groove end 528 of the cover device 400. According to an aspect of the present disclosure, the second groove end 528 and a majority of the groove 524 including a portion of the first groove end 526, are defined by groove walls 600 formed/defined by the outer surface 520 of the body 500 as illustrated in FIG. 6. It will be noted that at the first groove end 526, the groove walls 600 transition from the profile illustrated in FIG. 6 into the notches 530 at the first end face 506. According to an aspect of the present disclosure, the groove walls 600 are curved surfaces such that a cross-section of the groove 524 has a curved V-shape. According to another aspect of the present disclosure each groove wall 600 may have a radius curvature E in a range of 0.8 to 1.2 mm, and may preferably be 1.0 mm.

As illustrated in FIG. 6, the groove walls 600 extend from a joint edge 602 to an outermost portion of the outer surface 520, which defines the wall segments 522. The groove walls 600 are symmetric about the joint edge 602 that is located at a center of a trench defined by the groove 524. The cross-section of the groove(s) 524 is continued over a length of the groove(s) 524 from the second groove end 528 to the first groove end 526. As a result the wall segments 522 may be lobe-shaped as illustrated in FIGS. 5A, 5C, and 5D. However, it will be understood that other shapes (e.g. a flat angled wall, a wall extending 90° from a horizontal surface, an inwardly curved or parabolic surface, etc.) may be employed for the groove walls 600 such that the grooves 524 have different cross-sectional shapes (e.g. a straight V-shape, a square, a semi-circle, etc.). More generally, the groove walls 600 may have any shape, including a shape that define the grooves 524 to have asymmetrical cross-sections, that enables the grooves 524 to prevent the sutures 60 from moving radially relative to respective grooves 524.

According to an aspect of the present disclosure, the bodies 200, 500 of the cover devices 100, 400 described herein may be formed of a material that is elastically deformable. More specifically, the bodies 200, 500 may be formed from an elastically deformable sponge-like material that is compressible. For example the cover devices 100, 400 may be formed from certain types of thermoplastic materials such as ethyl vinyl acetate or other foam rubber materials. Further, the cover devices 100, 400 may be formed from a sheet of elastic material, such as a sheet formed from ethyl vinyl acetate. More specifically, the cover devices 100, 400 may be cut from the sheet alone, or cut from the sheet of elastic material and further molded into shape.

FIGS. 7A-7I illustrate a method of constructing and protecting a NAC 700 (hereafter referred to as “NAC constructing method 700”), according to an aspect of the present disclosure. In particular, FIG. 7A illustrates a process of designating a proposed NAC position 701 that includes drawing two concentric circles on a breast, such as the breast 10 illustrated in FIGS. 1 and 4 prior to reconstruction of the NAC 20. The outer circle corresponds to an outer diameter of an areola to be provided by a skin graft, such as the skin grafts 24 illustrated in FIGS. 1 and 4. The inner circle corresponds to a location of a base of a nipple to be constructed, such as the nipples 22 illustrated in FIGS. 1 and 4.

FIG. 7B illustrates a process of designating a flap outline 702, according to an aspect of the present disclosure. The flap outline 702 may be drawn on the breast to designate a line(s) for making an incision(s) to construct a nipple from a resulting flap (e.g. a skate flap, S flap, C-V flap, star flap, etc.). In general, a nipple may be reconstructed from an incised local flap of skin on the breast mound and fat tissue that are elevated together from a breast mound to create a protrusion. Typically, the incised local flap is closed by suturing opposing skin edges thereof together to form the nipple.

As illustrated in FIG. 7C, a flap 704 is created with a series of incisions made along the flap outline 702. A portion of the flap 704 will remain attached to an initial de-epithelialized bed 712a, which is created by making the incisions and pulling the flap 704 from the initial de-epithelialized bed 712a. FIG. 7C illustrates such a process of elevating a flap 704 on a pedicle core 706 for the NAC constructing method 700, according to an aspect of the present disclosure. The pedicle core 706 will remain connected to the initial de-epithelialized bed 712a and continue to provide a blood supply and vascularize the flap 704 once it is formed, thereby keeping the flap 704 alive. As illustrated in FIG. 7C, the exemplary flap 704 includes a cap 708 attached to the pedicle core 706 and wings 710 on opposite sides of the pedicle core 706.

FIG. 7D illustrates a process of wrapping the flap 704 around the pedicle core 706, according to an aspect of the present disclosure. In particular, the wings 710 are wrapped around the pedicle core 706 and the cap 708 is folded over the pedicle core 706 to form a cylindrical body and the tip of the nipple 22. Subsequent to wrapping the flap 704, the wings 710 are sutured to each other and the cap 708 is sutured to top edges of the wings 710 to thereby form a protruding tissue structure of the nipple 22 as shown in FIG. 7E.

FIG. 7E illustrates a process of designating additional skin 714 for removal from the breast 10, according to an aspect of the present disclosure. The circular dotted outline 715 illustrated in FIG. 7E is used to designate the location of one or more incisions to be made in order to remove the additional skin 714 that is radially between the nipple 22 and the circular dotted outline 715 (except for the portion of the flap attached to the initial de-epithelialized bed 712b). Once the additional skin 714 is removed, a circumferential skin edge, such as the circumferential skin edges 26 illustrated in FIGS. 1 and 4, is formed and an expanded de-epithelialized bed 712b (hereafter referred to as “bed 712b”) is exposed. The size and shape of the bed 712b corresponds to an areola to be formed by a skin graft such as the skin graft 24 illustrated in FIGS. 1 and 7E. One of ordinary skill in the art will recognize that a surgeon can form the circumferential skin edge 26 to have a diameter that corresponds to the second diameter B of a standardized version of the cover device 400 (or cover device 100).

FIG. 7F illustrates a process of positioning the skin graft 24, according to an aspect of the present disclosure. The skin graft 24 includes an incision cruciate 720, and is fitted into the bed 712b with the protruding tissue structure that is the nipple 22 projecting through the incision cruciate 720.

FIG. 7G illustrates a process of suturing the skin graft 24 in the bed 712b, according to an aspect of the present disclosure. In particular the skin graft 24 is sutured to the circumferential skin edge 26 and into the bed 712b by a plurality of sutures 30. In addition, several of the sutures 30 are left long (hereafter referred to as “long sutures 730”) and extend radially outward from the circumferential skin edge 26 as illustrated in FIG. 7G. The length of each long suture 730 is equal to or greater than at least two times a diameter of the skin graft 24, and may be extend past the outer diameter of the breast mound of the breast 10.

FIG. 7H illustrates a process of positioning the cover device 400 on the NAC 20, according to an aspect of the present disclosure. As illustrated in FIG. 7H, a cover device, for example the cover device 400 illustrated in FIGS. 4-5D, is positioned on the skin graft 24 of the NAC 20, and the nipple 22 of the NAC 20 is inserted into the channel 510. Prior to positioning the cover device 400 on the NAC 20, the cover device 400, which may have been gamma sterilized, is removed from a sterilized packaging. The tip of the nipple 22 is defined by the cap 708, which is sutured to the wings 708 of the flap 704. A location of the cap 708 along an axis of the channel 510 is between the first end face 506 and the second end face 508. More specifically, a height of the nipple 22 may be less than the height A of the cover device 400. Accordingly, the cover device 400 will protrude from the breast 400 past the tip of the nipple 22 and is thus configured to enclose and protect the nipple 22 from external forces (e.g. pressure/rubbing from garments).

As further illustrated in FIG. 7H, each groove 524 is aligned with a respective long suture 730 extending from the outer diameter of the skin graft 24/circumferential skin edge 26 of the breast 10. A number of long sutures 730 may be equal to a number of the grooves 524. According to an aspect of the present disclosure, there is one long suture 730 for each one of eight grooves 524. However, the cover device 400 may be secured to the NAC as discussed in more detail with reference to FIG. 7I, by more or less grooves 524 and corresponding long sutures 730. For example, the cover device 400 (or the cover device 100) may be provided with three, four, or six grooves 524 and a corresponding number of long sutures 730 may be provided during the suturing process illustrated in FIG. 7G.

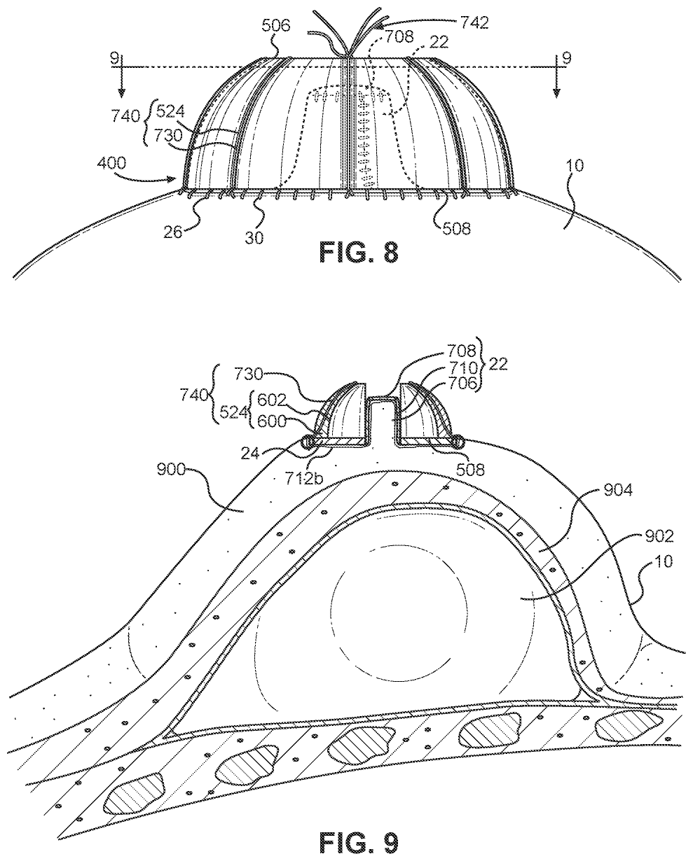

According to another aspect of the present disclosure, the number of long sutures 730 may not have to be equal to the number of grooves 524. The number of grooves 524 and sutures 730 must be such that a provided number of potential grooved suture combinations 740, which are illustrated in FIG. 7I and discussed below, are suitable to secure the cover device 400 (or the cover device 100) to the NAC 20 with the second end face 508 pressed against a surface of the skin graft 24 as discussed below with reference to FIGS. 8 and 9.

FIG. 7I illustrates a process of securing the cover device 400 to the NAC 20, according to an aspect of the present disclosure. FIG. 7I illustrates a plurality of the grooved suture combinations 740 in which a respective long suture 730 has been positioned within a corresponding second groove end 528, groove 524, first groove end 526, and notch 530. Accordingly, the long sutures 730 are positioned in corresponding grooves 524 between the wall segments 522. Further, the long sutures 730 are tied together inwardly relative to the notches 530 in a tie 742, and thereby apply pressure to the first end face 506. The applied pressure is transmitted through the body 500 to the second end face 508, which applies the pressure to the skin graft 24. Thus, the cover device 400 applies pressure to the skin graft 24 of the NAC 20. At the same time, the head 502, first end face 506, and inner surface 512, together with the tie 742 and portions of the long sutures 730 extending radially between the tie 742 and the first end face 506, protect the nipple 22 of the NAC 20.

FIG. 8 illustrates a side elevation of the breast 10 with a cover device 400 secured to the breast 10, according to an aspect of the present disclosure. The nipple 22 is shown within the cover device 400 by phantom lines. As illustrated in FIG. 8, the long sutures 730 are positioned in the grooves 524 and tied together in the tie 742. The second outer diameter C of the cover device 400 (i.e. of the second end face 508) may substantially correspond the circumferential skin edge 26/outer diameter of the skin graft 24. According to an aspect of the present disclosure, the second outer diameter C may be slightly less than the diameter of the circumferential skin edge 26, and as a result, the long sutures 730 may have to be moved/pulled radially inward relative to the circumferential skin edge 26/outer diameter of the skin graft 24, to be positioned within the second groove ends 528 and run through the grooves 524. According to another aspect of the present disclosure, the second outer diameter C may be slightly more than the diameter of the circumferential skin edge, such that portions of the long sutures 730 extending immediately from the circumferential skin edge 26/outer diameter of the skin graft 24 will be positioned (e.g. tucked) between the cover device 400 and the skin immediately surrounding the circumferential skin edge 26.

FIG. 9 illustrates a cross-sectional view of the breast 400 along section line 9-9 of FIG. 8. In particular, FIG. 9 illustrates the location of the skin graft 24 relative to fatty tissue 900 of the breast 10. As part of a breast reconstruction that may be performed in tandem with or separately from a mastectomy, an implant 902 may be inserted into the breast 10 through an incision (not shown) and may be partially covered by a pectoral muscle 904. Thus, the pectoral muscle 904 will be located within the breast 10 between the implant 902 and the fatty tissue 900. However, one of ordinary skill in the art will recognize that the implant 902 may not always be placed under the pectoral muscle 904. In particular, the implant 902 may be laid directly on top of the pectoral muscle 904 directly under the fatty tissue 900 of the skin of the breast 10 once the breast tissue has been removed. Subsequent to the procedure in which the implant 902 is inserted into the breast 10, the NAC 20 may be reconstructed at the apex of the breast 10 in a procedure as discussed above with reference to FIGS. 7A-7I. The cover devices 100, 400 of the present disclosure aid in the construction of both an areola and a nipple of a NAC as discussed in more detail below.

It is well known in the art to utilize skin grafts to form areolas in NAC reconstruction procedures as illustrated in FIGS. 7E-7F. A skin graft of a reconstructed NAC must be vascularized by blood vessels in the dermis of a de-epithelialized bed created in/on the breast. Successful revascularization will result in the blood vessels growing into the dermis of both a full or split-thickness skin graft and thereby attaching the skin graft to the de-epithelialized bed. This may be referred to as the skin graft “taking” to the de-epithelialized bed in which the skin graft is sustained by, and becomes a part of, the tissue and skin surrounding the location where the skin graft is applied.

In contrast, if the skin graft is not revascularized by the blood vessels in the de-epithelialized bed, the skin graft will die and be unusable. One cause of the skin graft not “taking” may be from blood accumulation in one or more small areas (e.g. pockets, divots, etc.) between the de-epithelialized bed and the skin graft. This can occur if a pressure pushing the skin graft onto the de-epithelialized bed is not evenly applied over the surface area of the skin graft.

It is well known in the art to employ split-thickness skin grafts or full-thickness skin grafts for NAC reconstruction procedures. A split-thickness skin graft is typically a raw surface with a portion of a dermis thereof removed. Split-thickness skin grafts may be taken from a donor site in slices with a device such as dermatome, and are often used for burns. A split-thickness skin graft, as compared to a full-thickness graft, may heal more rapidly. However, the donor site for a split thickness skin graft leaves a raw surface that has to heal by migration of skin cells from the residual skin appendages (sweat glands, hair follicles) remaining in a lower level of the dermis left behind by the taking of only an upper portion of the dermis (hence “split thickness”) with the skin graft.

A full-thickness skin graft is often taken from a part of the body with loose skin, and the donor site for the full-thickness skin graft is sutured together once the graft is removed. All fat underneath the skin graft is removed but a full-thickness of a respective dermis and epidermis remain. Revascularization of a full-thickness skin graft may be more difficult/take longer than a split-thickness skin graft generally. Thus, a potential for loss of a full-thickness skin graft (i.e. dying and having to be replaced), may be greater than for a split-thickness skin graft. However, as is typically the case, once an areola of a NAC has been reconstructed from a skin graft (either split or full-thickness), the resulting reconstructed areola may have to be colorized through a tattooing procedure to give the appearance of a normal/natural NAC. A reconstructed areola formed from a full-thickness skin graft may be thicker and more suitable (e.g., more sturdy, less sensitive, etc.) for such a tattooing procedure than a split-thickness skin graft.

Aspects of the cover devices 100, 400 of the present disclosure include a flat surface of second end faces 208, 508 that apply pressure evenly to the skin graft 24 upon the log sutures 730 being tied. As a result, the cover devices 100, 400 of the present disclosure may facilitate improved revascularization of either of a spilt- or full-thickness skin graft, relative to a method employing a bolster or syringe barrel, or other methods known in the art.

As illustrated in FIG. 9, the skin graft 24 is located between the bed 712b and the second end face 508 of the cover device 400. The bed 712b is a raw surface of the fatty tissue 900 from which the epidermis has been removed, and to which the skin graft 24 must successfully attach. Two long sutures 730 are also illustrated within respective grooves 524. When the long sutures 730 are tied together, in the tie 742, the cover device 400 presses the skin graft 24 towards the fatty tissue 900. As previously explained, the application of pressure is facilitated in part by the configuration of the grooves 524, the first groove ends 526, and notches 530 that provide the transitions from the outer surface 520 to the first end face 506. Said transitions promote uniform transmission and application of downward forces onto the first end face 506, which are evenly transmitted by the second end face 508 to the skin graft 24. Further, due to the flat, continuous, and even surface of the second end face 508; the second end face 508 applies pressure substantially evenly to the surface of the skin graft 24. As a result of the cover device 400 pressing the skin graft 24 towards the fatty tissue 900, the cover device 400 pushes out fluid between the skin graft 24 and the bed 712b, and facilitates vascularization by the blood vessels in the bed 712b of the skin graft 24. In addition, the cover device 400 may help prevent the skin graft 24 from becoming ulcerated.

As discussed above, it is imperative that a skin graft (split or full) of a reconstructed NAC be pressed evenly over a respective surface area on to a receiving bed. The cover devices 100, 400 accomplish this via flat second end faces 208, 508 being pressed down by tied long sutures 430, 730 which secure the cover devices 100, 400 to the NAC 20 as illustrated in FIGS. 3B, 7I, 8, and 9. In addition, as a result of the properties of the material from which the cover device 100 or cover device 400 are composed, the bodies 200, 500 of the cover devices 100, 400 each constitutes an elastic solid structure that maintains a respective shape if a force applied thereto remains static. Accordingly, the cover devices 100, 400 are more easily and reliably maintained in their respective forms, as applied immediately after nipple reconstruction, as well as a subsequent variable amounts of time, as compared to other methods such as the use of a bolster. Therefore, the attendant advantages of using a full-thickness skin graft may be obtained more reliably as compared to a bolster, for example, because the cover devices 100, 400 of the present disclosure can be utilized more easily to apply uniform and constant pressure for a time required for a full-thickness skin graft of a reconstructed NAC to take (become vascularized and attach to a bed).

Employing a skin graft (full or split) may provide advantages as compared to known NAC reconstruction methods in which a skin graft is not utilized. Unlike methods in which a skin graft is used, scars from reconstructing a nipple may remain visible through tattooed pigment for methods in which a skin graft is not utilized. This may occur as pigment uptake is not uniform in said scars. Hence the technique of providing a smooth uniform surface (healed skin graft) in order to obtain a non scar bearing surface for uniform and more cosmetically acceptable uptake of tattoo pigment is often preferred. However, the cover device 400 (or cover device 100) of the present disclosure may be used and provide advantages in NAC reconstructions where a skin graft is not used.

Other methods for NAC reconstruction may not implement the use of a skin graft to form an areola. However, as part of a majority, if not all NAC reconstructions, nipple reconstruction procedures commonly involve cutting a flap to wrap around a central core of fatty tissue, which then acts as a blood supply and ultimately as a “fill” within a healed nipple. As illustrated in FIGS. 3B, 7I, 8, and 9, cover devices 100, 400 of the present disclosure facilitate the construction of the nipple 22 of the NAC 20 as a result of inner surfaces 212, 512 of bodies 200, 500 that define channels 210, 510, which respectively can receive a nipple 22. The inner surfaces 212, 512 helps mold the nipple 22 by preventing it from slumping, while at the same time protects the nipple 22 since the channel 210, 510 is longer than the nipple 22 and thereby prevents a top of the nipple 22 from being exposed. Accordingly, cover devices 100, 400 of the present disclosure can be effectively used to construct and protect NACs whether a skin graft is used for the areola reconstruction or not.

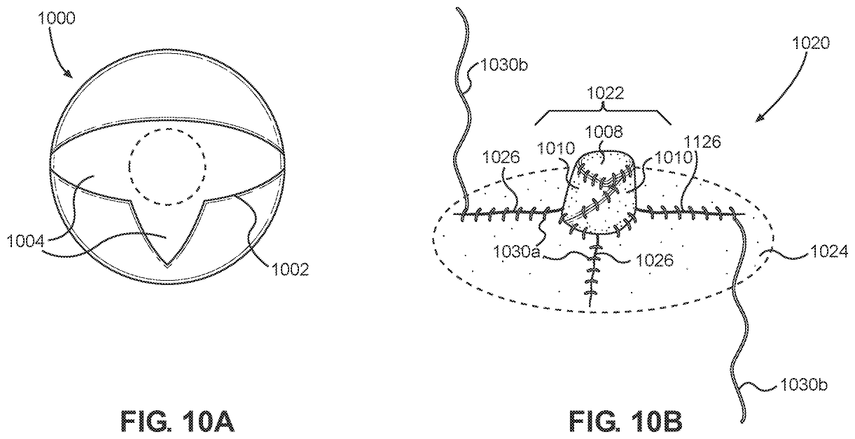

FIG. 10A illustrates an overhead view of a proposed position 1000 for a NAC 1020 for a procedure in which a skin graft is not utilized. FIG. 10A further illustrates a flap outline 1002 that once incised along, will provide a skate flap and correspond to a donor site 1004 on a breast mound. This type of NAC reconstruction (i.e. flap type reconstruction) may be employed where skin of a breast construction is lax enough to be pulled together without distorting a shape of the breast mound. It will be understood that other flap types (e.g. star, S-flap, etc.) may be used for this type of NAC reconstruction in which only a nipple is surgically reconstructed and an areola may be created through tattooing alone.

FIG. 10B illustrates a front perspective view of the NAC 1020 after a nipple 1022 has been reconstructed from a cap 1008 and wings 1010 that defined the skate flap provided by incision along the flap outline 1002 illustrated in FIG. 10A. Typical of this type of NAC reconstruction, in the absence of placing a skin graft for an areola, the donor site 1004 left by the elevation of skin/fat of the cap 1008 and wings 1010 is closed by suturing opposing skin edges 1026 together with sutures 1030a. Skin 1024 of the breast mound around the nipple 1022 will need to be tattooed in order to produce a facsimile of a different colored areola from surrounding breast skin. The tattoo pigment will need to be placed in to scars from the sutured incisions used to close the donor site 1004 left by the elevation of the cap 1008 and wings 1010.

As illustrated in FIG. 10B, the NAC 1020 is provided with long sutures for securing the cover device 400 (or cover device 100 could be used) to the skin 1024, which is designated in FIG. 10B by a dotted line 1025 surrounding the nipple 1022. The flat surface of the second end face 508 applies pressure evenly to the skin edges 1026 sutured together. Accordingly, attachment and healing of each pair of the skin edges 1026 extending from the nipple 1022 occurs under the same uniform conditions over a complete length respectively thereof as facilitated by the cover device 400 (or cover device 100) of the present disclosure.

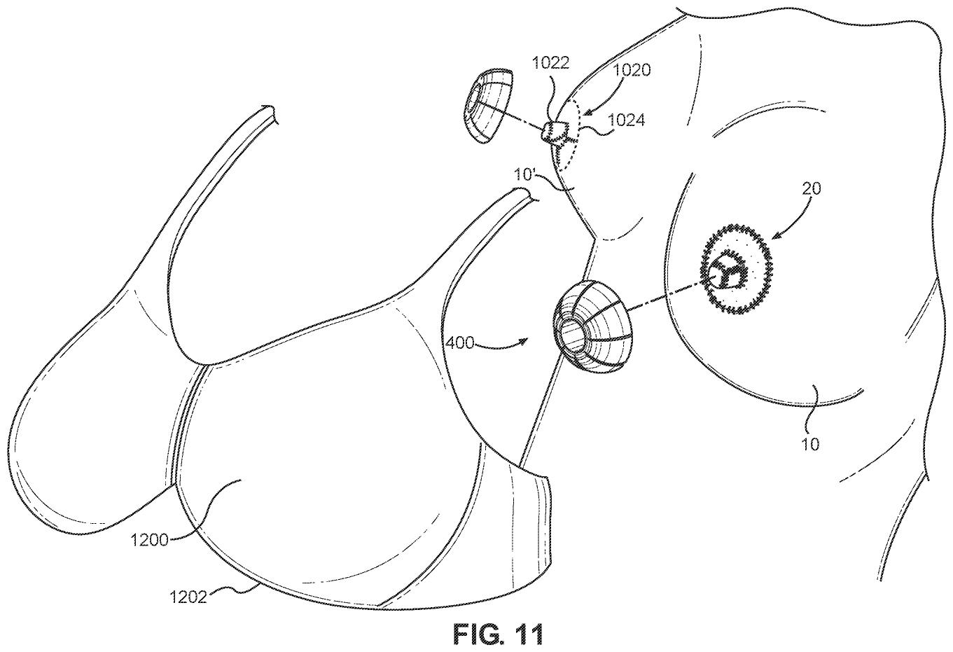

FIG. 11 illustrates a state of a pair of breasts 10, 10′ prior to being positioned in a breast support device 800 with cover devices 100, according to an aspect of the present disclosure. As can be seen, the breast 10 in the forefront of FIG. 11 includes the NAC 20 illustrated in FIGS. 7G-9, and the breast 10′ in the foreground of FIG. 11 includes the NAC 1020 illustrated in FIG. 10B. One of ordinary skill in the art will recognize that the same NAC reconstruction procedure may be performed for both breasts of patients as illustrated in FIGS. 1 and 4, or different NAC reconstructions may be performed as illustrated in FIG. 11. FIG. 11 includes both NAC types in order to demonstrate the applicability of the cover device 400 (or cover device 100) to different NAC reconstruction procedures.

The long sutures 730, 1030b and the cover device 400 (or cover device 100) may be removed 10 to 10 days after the NAC reconstruction procedure (nipple reconstruction in the case of the NAC 1020) so that each NAC 20, 1020 may be examined and evaluated from a healing (which in the case of NAC 20 would include taking of the skin graft 24) standpoint. FIG. 11 illustrates the state of each breast 10, 10′ after the long sutures 730 and cover device 400 (or cover device 100) have been removed and prior to the remaining sutures 30 being removed which would normally occur during such an examination. Once all the sutures 30 have been removed from each NAC 20, 1020 the cover device 400 (or cover device 100) may continue to be used to protect each NAC 20, 1020 without being fixedly secured to thereto, respectively.

During an examination of the type mentioned above, the same cover device 400 (or cover device 100) that was secured to each of the breasts 10, 10′ may be removed, cleaned, and again positioned on a respective NAC 20, 1020 to be held in place by a cup 802 of the breast support device 800. However, it will be understood that the cover device 400 (or cover device 100) is made of a material (e.g. ethyl vinyl acetate or the like) such that the cover device 400 (or cover device 100) is easily replaceable if desired. Thus, the same or a new cover device 400 (or cover device 100) may be utilized as a protective device to prevent injury to the newly constructed NAC 20, 720, especially the nipple 22,722 in the ensuing weeks until the NAC 20, 720 is deemed sufficiently healed to withstand normal “wear and tear” trauma from clothing and/or physical activity.

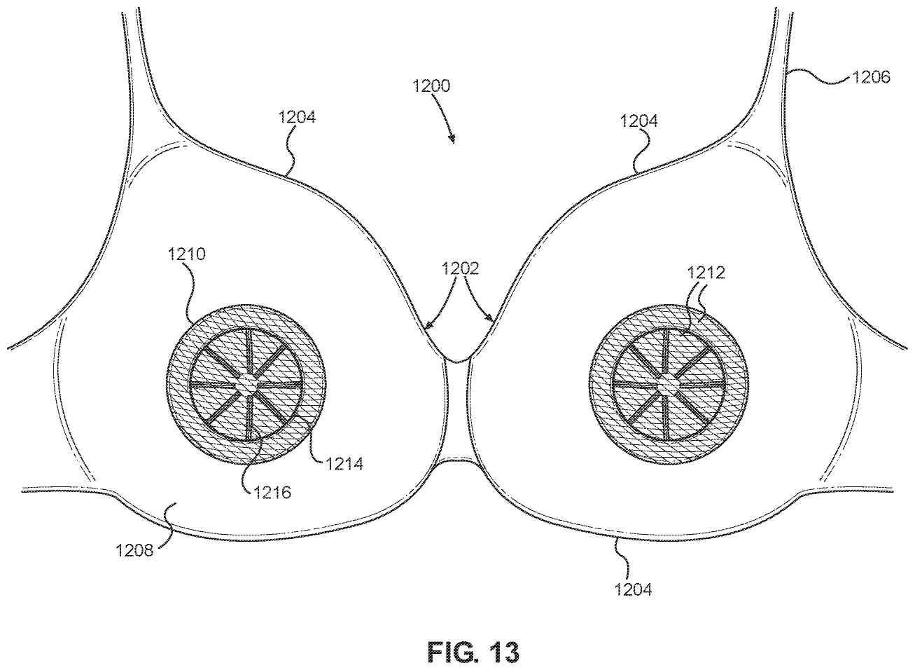

FIG. 13 illustrates a state of the pair of breasts 10, 10′ prior to being positioned in a breast support device 1200 with cover devices 100, according to an aspect of the present disclosure. The breast support device 1200 includes a cup 1202 configured to support each breast 10, and each cup 1202 may include a padded region 1208 extending immediately from an outer edge 1204 and straps 1205 of the breast support device 1200. The padded region 1208 may surround an intermediate edge 1210 that surrounds and attaches an elastic region 1212 to the padded region 1208. The elastic region 1212 may be made of a stretchable and breathable elastic fabric (e.g. polyester), and may be more flexible than the padded region 1208.

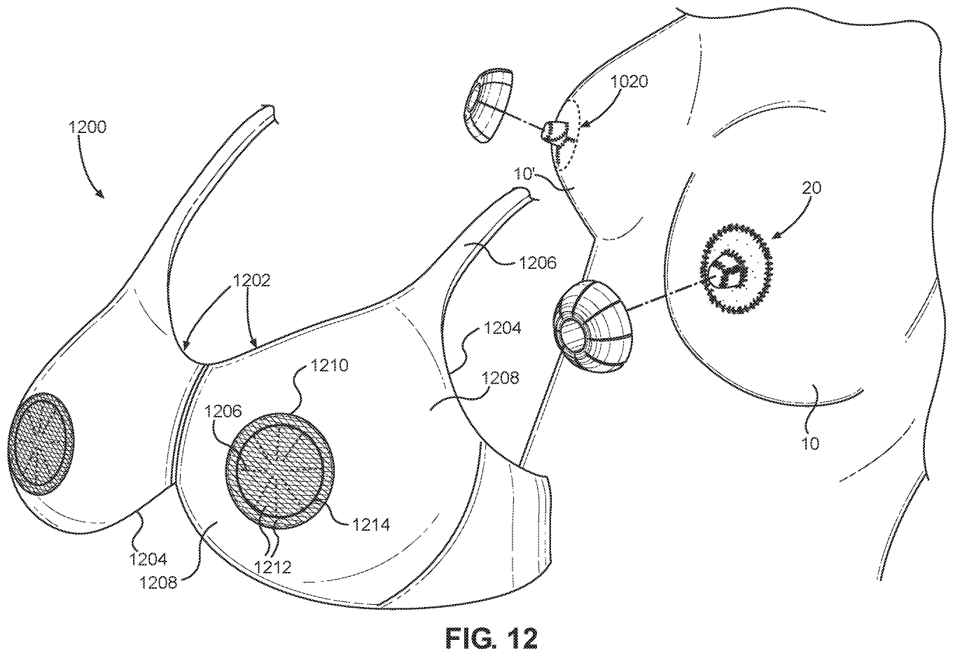

A diameter of the elastic region 1212 may be 1.0 to 2.0 cm larger than a diameter of a typical NAC 20, 720 that has been reconstructed. Further, the elastic region 1212 may include a seam 1214 that is concentric with the intermediate edge 1210 and has a diameter that is slightly greater than the typical reconstructed NAC 20, 720. A plurality of strips 1216 may extend from the seam 1214 towards the center of the elastic region 1212 and have a length and cross-section corresponding to the length and cross-section of the grooves 524 of the cover device 100. The strips 1216 will function to help secure the cover device 400 (or cover device 100) in place once the long sutures 730, 730b are removed such that the nipple 22,722 of the NAC 20, 720 is not solely responsible from holding the cover device 400 (or cover device 100) in place surrounding the nipple 22,722. Therefore, the breast support device 1200 illustrated in FIG. 12 is constructed to hold the cover device 400 (or cover device 100) in place once the long sutures 730, 730b have been removed.

FIG. 10 illustrates an elevation view of an inner cup surface of the breast support device 1200, according to an aspect of the present disclosure. As illustrated in FIG. 10, the strips 1216 extend from an inner surface of the elastic region 1212. Thus, the cover device 400 (or cover device 100) can be fitted on to the strips 1216 and together with the breast support device 1200 can be positioned and secured on a patient with the straps 1205 of the breast support device 1200. Alternatively, the cover device 400 (or cover device 100) can be positioned on the breast 10 (or breasts), and the breast support device 1200 can be positioned on the cover device(s) 100. According to an aspect of the present disclosure, the strips 1216 may extend from the inner cup surface of the breast support device 1200 and fit into the grooves 524 such that the outer surface 520 of the cover device 400 (or cover device 100) is flush with the inner surface of the elastic region 1212. According to another aspect of the present disclosure, the strips 1216 may be sized to fit into the grooves 524 such that a gap is provided between the outer surface 520 of the cover device 400 (or cover device 100) is flush with the inner surface of the elastic region 1212.

It will be appreciated that the foregoing description provides examples of the disclosed cover device and techniques for applying the cover device. These examples given above are merely illustrative and are not meant to be an exhaustive list of all possible designs, aspects, applications or modifications of the disclosure. Further, it is contemplated that other implementations of the disclosure may differ in detail from the foregoing examples.

For example, exemplary cover devices described herein include grooves that a formed in an outer surface of a body and include notches formed in a first end face. However, other versions of a cover device according to the present disclosure may be provided with grooves that include body grooves/portions in an outer surface or notches. For example, grooves of an example of cover device 100 of FIGS. 1-3B may include just notches 230 extending from a perimeter edge 206 to an inner edge 207 of a first end face 205. In another examples of cover device 100, some grooves may include notches 230 and body grooves 225, whereas other grooves only include body grooves 225 or notches 230. In still other examples of a cover device of the present disclosure, an outer surface and a first end face may define uninterrupted surfaces. In this example, no grooves may be defined by a body and end face as warranted by particular conditions attendant to a subject on which the cover device will be used.

Furthermore, example cover devices of the present disclosure are described herein and illustrated in some of the drawings as being secured to a NAC by sutures. However, additional methods of securement may be used, especially in the context of a post-operative situation. Thus, in some examples, a second end face (i.e., a surface of a cover device that presses a skin graft defining an areola of a NAC) may be provided with a layer of adhesive (e.g., TEGADERM).

All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

We claim:

1. A cover device comprising:
a body including an inner surface and an outer surface, each of the inner surface and the outer surface extending from a first end face of the body to a second end face of the body,
wherein the inner surface defines a channel extending through the body from the first end face to the second end face,
wherein the body defines a plurality of grooves extending from the first end face to the second end face, portions of each groove of the plurality of grooves being defined by the inner surface, the first end face, the outer surface, and the second end face,
wherein each of the body, the inner surface, and the outer surface are configured to elastically deform relative to at least an axis of the channel, and
wherein the second end face defines a substantially flat surface configured to evenly distribute an external force applied to at least one of the outer surface and the first end face.

2. The cover device of claim 1, wherein each groove includes a body groove and a notch, the notch being formed in the first end face and extending from a respective body groove.

3. The cover device of claim 2, wherein the outer surface defines a plurality of wall segments between adjacent body grooves of the plurality of grooves, and wherein each wall segment defines a surface that is curved outwardly from an outer circumference of the first end face to an outer circumference of the second end face.

4. The cover device of claim 2, wherein each notch extends to the inner surface from a body groove end of the respective body groove, the body groove end disposed at a perimeter edge of the first end face.

5. The cover device of claim 2, wherein each notch includes walls that extend from the first end face and converge to a respective joint edge.

6. The cover device of claim 6, wherein joint edges of the notches extend parallel to the second end face.

7. The cover device of claim 6, wherein joint edges of the notches extend at an angle relative to a plane of the second end face.

8. The cover device of claim 1, wherein the body is formed from a thermoplastic elastomer.

9. The cover device of claim 2,
wherein a body groove of each of the plurality of grooves extends from a body groove end formed in the second end face and is defined by a pair of groove walls, and
wherein each pair of groove walls is symmetric about a joint edge that is located at a center of a trench defined by a respective body groove.

10. A method of constructing and protecting nipple areola complex of a breast, the method comprising:
suturing a skin graft corresponding to an areola of the nipple/areola complex in a de-epithelialized bed surrounding a nipple of the nipple/areola complex;
leaving a plurality of long sutures during the suturing, each of the plurality of long sutures extending over a length from an outer circumference of the skin graft that is equal to at least twice the diameter of the skin graft;
positioning a cover device relative to the nipple/areola complex such that:
the nipple is positioned within a channel defined by an inner surface of the cover device with a cap of the nipple positioned between a first end face and a second end face of the cover device, and
the second end face abuts a surface of the skin graft;
positioning each of the plurality of long sutures in a respective groove defined by a body of the cover device; and
tying the plurality of long sutures together at the first end face such that the cover is secured to the nipple/areola complex with the second end face in fixed abutment with the surface of the skin graft,
wherein each groove includes a body groove formed in an outer surface of the body and a notch formed in the first end face, the notch extending from a respective body groove to the inner surface.

11. The method of constructing and protecting nipple areola complex of claim 10,
wherein the body of the cover device includes the inner surface and the outer surface extending from the first end face to the second end face, and
wherein each of the body, the inner surface, and the outer surface are configured to elastically deform relative to at least an axis of the channel.

12. The method of constructing and protecting nipple areola complex of claim 11, wherein the body is formed from a thermoplastic elastomer.

13. The method of constructing and protecting nipple areola complex of claim 10, wherein each body groove defined by the outer surface of the cover device extends from a first groove end formed at a perimeter edge of the first end face to a second groove end formed in the second end face.

14. The method of constructing and protecting nipple areola complex of claim 13, wherein tying the plurality of long sutures includes each suture following a path of a respective groove body and notch such that a downward force is applied to the first end face by each suture.

15. The method of constructing and protecting nipple areola complex of claim 10, further comprising:
removing the long sutures and the cover device from the nipple/areola complex;
cleaning the cover device;
positioning the cover device on the nipple/areola complex; and
positioning a breast support device on the breast,
wherein the cover device is configured to be secured to the nipple/areola complex by the breast support device.

16. A cover device comprising:
a body including an inner surface and an outer surface, each of the inner surface and the outer surface extending from a first end face of the body to a second end face of the body,
wherein the inner surface defines a channel extending through the body from the first end face to the second end face,
wherein the second end face defines a substantially flat surface configured to evenly distribute an external force applied to at least one of the outer surface and the first end face,
wherein the body defines a plurality of grooves extending from the first end face to the second end face, portions of each groove of the plurality of grooves being defined by the inner surface, the first end face, the outer surface, and the second end face,
wherein each groove includes a body groove defined in the outer surface and a notch formed in the first end face extending from a respective body groove.

17. The cover device of claim 16, wherein each notch extends to the inner surface from a body groove end of the respective body groove, the body groove end disposed at a perimeter edge of the first end face, and wherein each notch includes walls that extend from the first end face and converge to a respective joint edge.

18. The cover device of claim 17, wherein joint edges of the notches extend parallel to the second end face.

19. The cover device of claim 17, wherein joint edges of the notches extend at an angle relative to a plane of the second end face.

20. The cover device of claim 16, wherein the body is formed from a thermoplastic elastomer.
